(19) United States
(12) Patent Application Publication
(10) Pub. No.: US 2024/0024665 A1
(43) Pub. Date: Jan. 25, 2024

(54) DEFORMABLE SPINAL CORD STIMULATION DEVICE AND RELATED SYSTEMS AND METHODS

(71) Applicant: NeuroOne Medical Technologies Corporation, Eden Prairie, MN (US)

(72) Inventors: Maria Vomero, Astoria, NY (US); Samuel Ong, San Francisco, CA (US); Maria Porto Cruz Westermann, Düsseldorf (DE); Hijaz Haris, Plymouth, MN (US); Steve Mertens, Plymouth, MN (US); Dave Rosa, Eden Prairie, MN (US); Camilo Diaz-Botia, Monte Sereno, CA (US); Alfonso Chavez, San Jose, CA (US)

(21) Appl. No.: 18/358,859

(22) Filed: Jul. 25, 2023

Related U.S. Application Data
(60) Provisional application No. 63/391,818, filed on Jul. 25, 2022.

(51) Int. Cl. A61N 1/05

(52) U.S. Cl. CPC A61N 1/0553

(57) ABSTRACT

Various deformable thin film spinal cord stimulation devices that are deformable to conform to the shape and/or movement of the target spinal cord. Each device embodiment has an electrode body with at least one deformation section disposed longitudinally within the electrode body and at least one electrode contact. Some implementations have a distal structure that can be formed into a pusher receiving structure such as a pocket or a collar.

### CROSS-REFERENCE TO RELATED APPLICATION(S)

This application claims the benefit under 35 U.S.C. § 119(e) to U.S. Provisional Application 63/391,818, filed Jul. 25, 2022 and entitled “Deformable Spinal Cord Stimulation Device and Related Systems and Methods,” which is hereby incorporated herein by reference in its entirety.

### FIELD

The various embodiments herein relate to devices for stimulating the spinal cord and/or peripheral nerves and related systems and methods.

### BACKGROUND

Electrical stimulation of the spinal cord can result in pain reduction and/or elimination. Medical devices having electrodes (also referred to as “stimulators” or “leads”) are often implanted near the spinal column to provide pain relief for chronic intractable pain. The electrodes stimulate tissue within the spinal column to reduce pain sensations at other parts of the body. The stimulation signals applied can be optimized for pain reduction or elimination depending on the location of the pain.

Known spinal cord stimulation devices are typically percutaneous leads 10 or paddle leads 12, as the exemplary devices depict in FIG. 1. Both device types are mainly made of silicone rubber and platinum-iridium. One disadvantage of these known lead types is their inability to conform to and flex or deform with the spinal cord to which they are attached. That is, the known devices have high bending stiffness and lack of conformability with the target tissue that result from the combination of (1) the soft material with which they are made and (2) the substrate thickness (i.e., 1 mm to 2 mm) necessary to achieve mechanical robustness and long-term reliability of the implant.

Other limitations of the known spinal cord stimulation devices will also become evident in the Detailed Description.

There is a need in the art for improved thin film spinal cord stimulation devices and related systems and methods.

### BRIEF SUMMARY

Discussed herein are various deformable spinal cord stimulation devices and methods, including various devices having at least one deformation section disposed along a length of the electrode body.

In Example 1, a spinal cord stimulation device comprises an elongate thin film lead body, and a thin film electrode body disposed at one end of the elongate thin film lead body. The thin film electrode body comprises at least one deformation section disposed longitudinally through the electrode body such that the at least one deformation section is parallel with a longitudinal axis of the lead body, and at least two contacts disposed on the electrode body.

Example 2 relates to the device according to Example 1, wherein the at least two contacts comprises at least twelve contacts.

Example 3 relates to the device according to Example 1, wherein the at least one deformation section comprises at least two deformation sections.

Example 4 relates to the device according to Example 1, wherein the at least one deformation section comprises three deformation sections.

Example 5 relates to the device according to Example 1, wherein the thin film electrode body comprises a distal flap disposed at a distal end of the thin film electrode body.

Example 6 relates to the device according to Example 5, further comprising at least one distal deformation section comprising a first end disposed at a distal end of the at least one deformation section and a second end disposed at a side of the electrode body.

Example 7 relates to the device according to Example 6, wherein the second end of the at least one distal deformation section is disposed between the distal flap and the distal end of the thin film electrode body.

Example 8 relates to the device according to Example 5, wherein the flap is moveable between a flat configuration and a pocket configuration.

Example 9 relates to the device according to Example 5, wherein the flap is moveable between a flat configuration and a collar configuration.

In Example 10, a spinal cord stimulation device comprises an elongate thin film lead body, and a thin film electrode body disposed at one end of the elongate thin film lead body. The thin film electrode body comprises at least two deformation sections disposed longitudinally through a middle portion of the electrode body, at least one first contact disposed between a first outer edge of the electrode body and a first of the at least two deformation sections, at least one second contact disposed between the first and a second of the at least two deformation sections, and at least one third contact disposed between the second of the at least two deformation sections and a second outer edge of the electrode body, wherein the first and second outer edges are moveable in relation to each other via the at least two deformation sections such that the electrode body is laterally conformable to a shape of a target spinal cord.

Example 11 relates to the device according to Example 10, further comprising at least one pair of notches defined in the first and second sides of the electrode body, whereby the electrode body has increased lateral and longitudinal flexibility.

Example 12 relates to the device according to Example 10, wherein the first and second outer edges are rotatable around a longitudinal axis of the electrode body.

Example 13 relates to the device according to Example 10, wherein the thin film electrode body comprises a distal flap disposed at a distal end of the thin film electrode body,

Example 14 relates to the device according to Example 13, wherein the flap is moveable between a flat configuration and a pocket configuration or a collar configuration.

In Example 15, a spinal cord stimulation device comprises an elongate thin film lead body and a thin film electrode body disposed at one end of the elongate thin film lead body. The thin film electrode body comprises at least three deformation sections disposed longitudinally through the electrode body such that each of the at least three deformation sections is parallel with a longitudinal axis of the lead body, at least two contacts disposed on the electrode body, and first and second outer edges moveable in relation to each other via the at least three deformation sections such that the electrode body is laterally conformable to a shape of a target spinal cord. The stimulation device further comprises a distal flap disposed at a distal end of the thin film electrode body, wherein the flap is movable between a flat configuration and a pocket configuration or a collar configuration.

Example 16 relates to the device according to Example 15, further comprising at least one distal deformation section comprising a first end disposed at a distal end of one of the at least three deformation sections and a second end disposed at one of the first and second outer edges.

Example 17 relates to the device according to Example 16, wherein the second end of the at least one distal deformation section is disposed between the distal flap and the distal end of the thin film electrode body.

In Example 18, a method of implanting a deformable spinal cord stimulation device comprises preparing the deformable spinal cord stimulation device for implantation, wherein the deformable spinal cord stimulation device comprises an elongate thin film lead body and a thin film electrode body disposed at one end of the elongate thin film lead body, the thin film electrode body comprising at least one deformation section disposed longitudinally through the electrode body such that the at least one deformation section is parallel with a longitudinal axis of the lead body and at least two contacts disposed on the electrode body. The device further comprises a distal flap disposed at a distal end of the thin film electrode body, wherein the flap is movable between a flat configuration and a pusher receiving configuration. The method further comprises urging the distal flap into the pusher receiving configuration, inserting a distal end of a pusher device into the pusher receiving configuration, and urging the deformable spinal cord stimulation device into a target area of a patient's spinal cord with the pusher device.

Example 19 relates to the method according to Example 18, wherein the at least one deformation section comprises at least two deformation sections.

Example 20 relates to the method according to Example 18, wherein the pusher receiving configuration comprises a pocket configuration or a collar configuration.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a perspective view of certain known percutaneous spinal cord stimulation devices and paddle spinal cord stimulation devices.

FIG. 2A is a top view of target area of the spinal cord for the various spinal cord simulation device embodiments disclosed or contemplated herein.

FIG. 2B is a cross-sectional side view of the target area of FIG. 2A.

FIG. 2C is a schematic perspective view of a standard stimulation device disposed adjacent to a spinal cord.

FIG. 2D is a schematic perspective view of a deformable spinal cord stimulation device positioned against a spinal cord, according to one embodiment.

FIG. 3 is a perspective view of a deformable spinal cord stimulation device, according to one embodiment.

FIG. 4A is a top view of deformable spinal cord stimulation device, according to another embodiment.

FIG. 4B is a cross-sectional side view of the deformable spinal cord stimulation device of FIG. 4A, according to one embodiment.

FIG. 4C is a cross-sectional side view of the deformable spinal cord stimulation device of FIG. 4A in use, according to one embodiment.

FIG. 5A is a top view of deformable spinal cord stimulation device, according to a further embodiment.

FIG. 5B is a cross-sectional side view of the deformable spinal cord stimulation device of FIG. 5A, according to one embodiment.

FIG. 5C is a cross-sectional side view of the deformable spinal cord stimulation device of FIG. 5A in use, according to one embodiment.

FIG. 6A is a top view of deformable spinal cord stimulation device, according to yet another embodiment.

FIG. 6B is a cross-sectional side view of the deformable spinal cord stimulation device of FIG. 6A, according to one embodiment.

FIG. 6C is a cross-sectional side view of the deformable spinal cord stimulation device of FIG. 6A in use, according to one embodiment.

FIG. 7A is a top view of deformable spinal cord stimulation device, according to an alternative embodiment.

FIG. 7B is a cross-sectional side view of the deformable spinal cord stimulation device of FIG. 7A in use, according to one embodiment.

FIG. 8A is a top view of deformable spinal cord stimulation device with a coating, according to another alternative embodiment.

FIG. 8B is a cross-sectional side view of the deformable spinal cord stimulation device of FIG. 8A in use, according to one embodiment.

FIG. 9A is a cross-sectional side view of a deformable spinal cord stimulation device, according to one embodiment.

FIG. 9B is a cross-sectional side view of another deformable spinal cord stimulation device, according to another embodiment.

FIG. 9C is a cross-sectional side view of a deformable spinal cord stimulation device with a coating, according to one embodiment.

FIG. 10A is a top view of deformable spinal cord stimulation device, according to one embodiment.

FIG. 10B is another top view of the deformable spinal cord stimulation device of FIG. 10A, according to one embodiment.

FIG. 100 is another top view of the deformable spinal cord stimulation device of FIG. 10A, according to one embodiment.

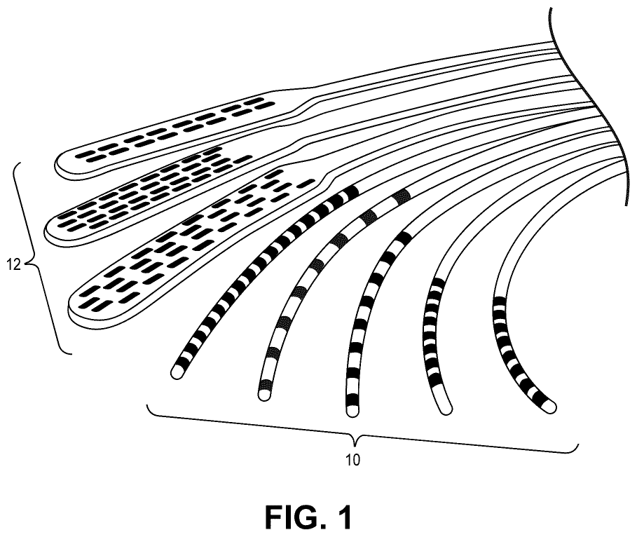

FIG. 11A is a top view of a distal tip of a deformable spinal cord stimulation device, according to one embodiment.

FIG. 11B is another top view of the deformable spinal cord stimulation device of FIG. 11A in which the tip has been formed into a pocket, according to one embodiment.

FIG. 11C is another top view of the deformable spinal cord stimulation device of FIG. 11A in which a pusher has been positioned within the pocket, according to one embodiment.

FIG. 11D is a side view of the deformable spinal cord stimulation device as shown in FIG. 11C, according to one embodiment.

FIG. 12A is a top view of a distal tip of another deformable spinal cord stimulation device, according to another embodiment.

FIG. 12B is another top view of the deformable spinal cord stimulation device of FIG. 12A in which the tip has been formed into a pocket, according to one embodiment.

FIG. 12C is another top view of the deformable spinal cord stimulation device of FIG. 12A in which a pusher has been positioned within the pocket, according to one embodiment.

FIG. 12D is a side view of the deformable spinal cord stimulation device as shown in FIG. 12C, according to one embodiment.

FIG. 13A is a top view of a distal tip of another deformable spinal cord stimulation device, according to a further embodiment.

FIG. 13B is another top view of the deformable spinal cord stimulation device of FIG. 13A in which the tip has been formed into a collar, according to one embodiment.

FIG. 13C is another top view of the deformable spinal cord stimulation device of FIG. 13A in which a pusher has been positioned within the collar, according to one embodiment.

FIG. 13D is a side view of the deformable spinal cord stimulation device as shown in FIG. 13C, according to one embodiment.

FIG. 14A is a top view of a distal tip of yet another deformable spinal cord stimulation device, according to another embodiment.

FIG. 14B is another top view of the deformable spinal cord stimulation device of FIG. 14A in which the tip has been formed into a collar, according to one embodiment.

FIG. 14C is another top view of the deformable spinal cord stimulation device of FIG. 14A in which a cap has been positioned over the collar and a pusher has been positioned within the collar, according to one embodiment.

FIG. 14D is another top view of the deformable spinal cord stimulation device of FIG. 14A in which the pusher has been urged distally such that distal tip is no longer positioned within the cap, according to one embodiment.

FIG. 14E is a side view of the deformable spinal cord stimulation device as shown in FIG. 14C, according to one embodiment.

FIG. 15A is a top view of a distal end of a pusher device, according to one embodiment.

FIG. 15B is another top view of the pusher device of FIG. 15A in which a deformable spinal cord stimulation device has been attached via the flap, according to one embodiment.

FIG. 15C is a side view of the pusher device as shown in FIG. 15B, according to one embodiment.

### DETAILED DESCRIPTION

The various embodiments disclosed or contemplated herein relate to improved systems, devices, and methods, and various components thereof, for stimulating the spinal cord in the human body. In certain exemplary implementations, each of the various stimulation systems and devices incorporates thin-film technology and a flexible electrode body that allows for conformity of the body to the spinal cord and movement of the electrode body to mirror the movement of the spinal cord. Some embodiments relate to chronically implantable stimulation devices that can remain implanted for five years or more. The various implementations are ultra-low profile devices, wherein each such device has a thickness ranging from about 25 μm to about 200 μm (and such ultra-low profile devices with a coating or overmold can have a thickness up to 500 μm). Alternatively, each of the various device embodiments herein can have a thickness ranging from about 50 μm to about 75 μm. In contrast, known spinal cord stimulation devices generally have an average thickness of about 1.5 mm (and typically greater with a coating or overmold).

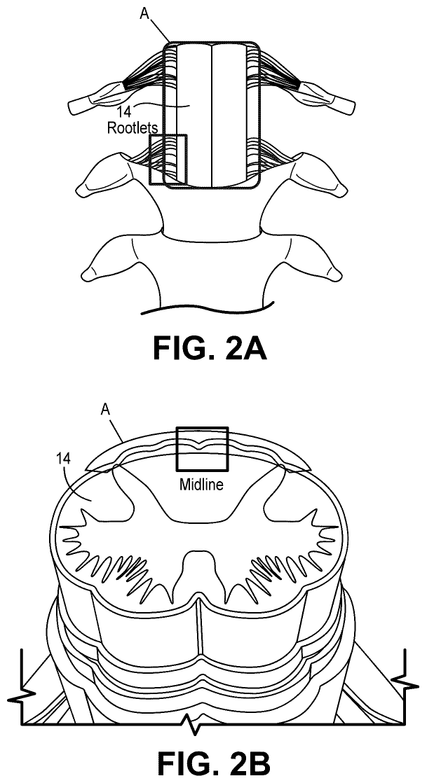

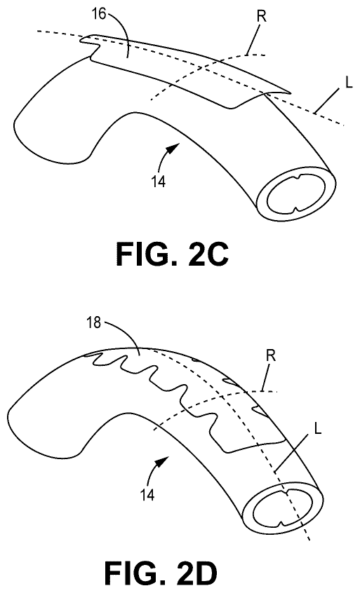

In accordance with certain implementations, the various stimulation device embodiments herein can be positioned over and against the spinal cord 14 as shown in FIGS. 2A-B and 2D. More specifically, as shown in FIGS. 2A-2B, the various device embodiments herein can be positioned over the target area depicted by the area A such that the electrode body (including any of bodies 22, 32, 42, 52, 72 discussed in further detail below) covers at least the area identified as the target area depicted by the area A. Alternatively, other embodiments include an electrode body (such as body 62, for example) that is positioned over a portion of the spinal cord on one side or the other of the midline such that the electrode body covers at least a portion of the area A. As such, as shown in FIG. 2D, the deformability of the various body embodiments herein (including bodies 22, 32, 42, 52, 62 72) can allow for those bodies to bend around and thus conform to the outer curvature of the spinal cord 14 and/or flex longitudinally along with the spinal cord 14 such that the bodies maintain better contact with the spinal cord 14 than non-deformable devices. More specifically, in contrast to a known stimulation device 16 that is not deformable radially (as represented by line R) or longitudinally (as represented by line L) as shown in FIG. 2C, the various embodiments herein as represented by the device 18 in FIG. 2D are deformable both radially as shown with line R such that the device embodiments conform to the radial curvature of the spinal cord and longitudinally as shown with line L such that the embodiments conform to the longitudinal curvature of the spinal cord 14 as shown. In certain implementations, any of the devices herein can conform to the spinal cord surface and/or movements while in contact with the spinal cord surface or even if the device is disposed within the epidural space but not in direct contact with the spinal cord surface. More specifically, the various device embodiments are conformable both anatomically and mechanically. In other words, in some embodiments, any device implementation herein may physically interface with/touch the spinal cord (or the dura, to be more specific), while in other embodiments, any device herein may be separated from the spinal cord/dura by a thin layer of fat within the epidural space. Regardless, the various device embodiments herein can conform to the anatomy and/or follow the movements thereof. These benefits of the deformable thin film device embodiments disclosed herein will be discussed in additional detail below.

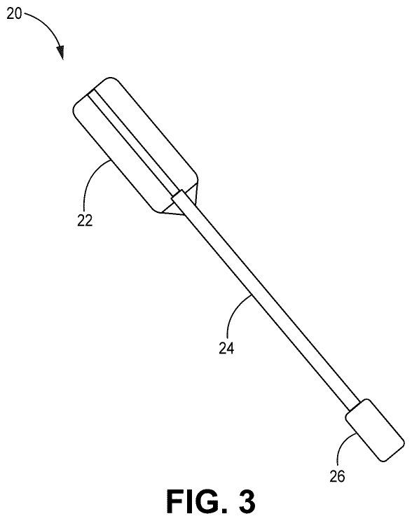

FIG. 3 depicts a deformable spinal cord stimulation device 20 according to one embodiment. The device 20 has an electrode body 22 (also referred to as a “contact body” or “paddle”) on which the one or more electrodes (not shown) are disposed, a lead body (also referred to as a “tail”) 24, and a connection component (also referred to as a “proximal connection component,” “connector,” or “proximal connector”) 26 to which the external electrical source is coupled.

In accordance with various implementations, the device 20 can be considered a hybrid between a percutaneous lead and a paddle lead. For example, the device 20 can be implanted percutaneously but can offer a paddle-like coverage of the spinal cord (due to a width that is essentially equivalent to two or more percutaneous leads placed in parallel along the spine).

Each of the components of the device 20 (including the electrode body 22, the lead body 24, and the proximal connector 26) can be thin film components, with some of those components or portions of the device 20 being coated in any device embodiments herein with thin layers of silicone rubber (wherein various embodiments of the device having such rubber layers can have a total thickness of no more than 0.5 mm). For purposes of this application, the term “thin film” can mean a microscopically thin layer of material that is deposited onto a metal, ceramic, semiconductor or plastic base, or any device having such a component. Alternatively, for purposes of this application, it can also mean a component that is less than about 0.127 mm (0.005 inches) thick and contains a combination of conductive and dielectric layers or a device that has one or more such components, wherein the components can be combined in a stacked or layered configuration in the device. Finally, it is also understood, for purposes of this application, to have the definition that is understood by one of ordinary skill in the art.

Further, the various non-conducting thin-film components of the device 20 (and any other device embodiment herein) can be made of polyimide (“PI”), parylene C, liquid crystal polymer (“LCP”), or similar materials. Further, the conductive materials used in the device 20 (for the contacts and traces, for example) can be any one or more of platinum, platinum iridium, iridium oxide, titanium, or any other known conductive metal for use in spinal or neural probe devices.

Any of the individual components, mechanisms, features, functionality, and/or dimensions of the device embodiment of FIG. 3 described in detail above can be incorporated into any of the other system embodiments discussed below. Similarly, any of the individual components, mechanisms, features, functionality, and/or dimensions of any of the device embodiments described in detail below can be incorporated into the embodiment of FIG. 3 or any of the other embodiments discussed below.

According to one embodiment, the various device embodiments herein can be constructed in the following fashion. Two layers of non-conductive thin-film components are provided, with the conductive components disposed therebetween. In certain implementations, once the three portions are combined and attached to each other according to any known method, the top non-conductive layer can be etched at the desired locations to create access to the contacts. Alternatively, any known method of construction can be used.

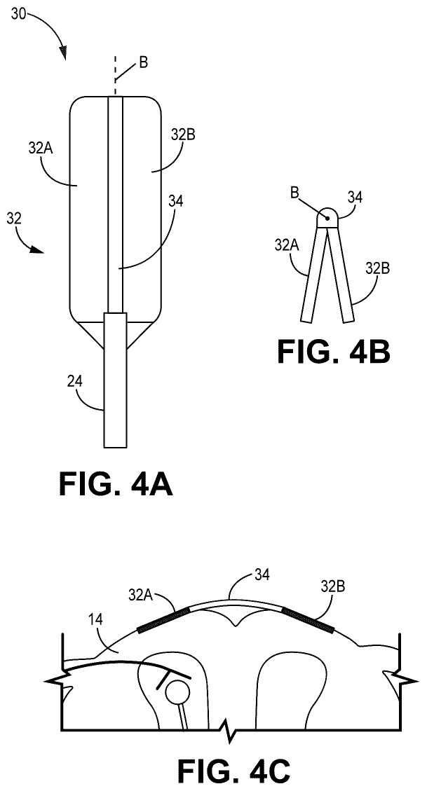

Various spinal cord stimulation device implementations are depicted in FIGS. 4A-100. These embodiments have structural features or characteristics that increase the flexibility of the electrode body (along with the lead body), thereby allowing the body to be capable of conforming to and moving with the target spinal cord when the body is positioned against or adjacent to that spinal cord.

According to one exemplary embodiment, FIG. 4A depicts a device 30 having an electrode body 32 that has a section (“middle section,” “deformation section,” or “thin section”) 34 disposed through the middle of the body 32 as shown that is thinner than the rest of the body 32. More specifically, the thin section 34 extends along the length of and substantially through the middle of the electrode body 32 as shown such that the thin section 34 is substantially parallel with the lead body 24. The position of the thin section 34 allows the body 32 to more readily bend or otherwise deform along the longitudinal middle of the body 32 (at the deformation section 34) such that both sides 32A, 32B of the body 32 on opposing sides of the thin section 34 can be urged toward each other around an axis B created by the thin section 34. Each of the sides 32A, 32B contains at least one contact (not shown) disposed thereon, with various embodiments having a plurality of contacts (not shown) disposed on each side 32A, 32B.

For example, FIG. 4B depicts the electrode body 32 along the longitudinal axis of the body 32 such that the longitudinal axis extends out of the page. As shown, the two sides 32A, 32B are deformed toward each other around the axis B created by the deformation section 34.

In one implementation, the deformation section 34 has a thickness ranging from about 15 micrometers (μm) to about 25 μm. The body 32, according to one embodiment, can have a thickness ranging from about 50 μm to about 75 μm. Alternatively, the body 32 can have a thickness of up to about 100 μm.

In addition, the electrode body 32 can have a width ranging from about 5 mm to about 8 mm, the thin section 34 can have a width ranging from about 1 mm to 3 mm, and the lead body 24 can have a width ranging from about 1 mm to about 3 mm.

FIG. 4C depicts the device 30 in use. More specifically, the electrode body 32 is positioned on the spinal cord 14 so that the body 32 is positioned over an area substantially similar to the area A depicted in FIGS. 2A and 2B as discussed above. Further, the body 32 is deformed at the deformation section 34 such that the two sides 32A, 32B conform to the shape of the spinal cord 14 such that the sides 32A, 32B are positioned in contact with the spinal cord 14 on both sides of the midline as shown, thereby ensuring that the body 32 is in better contact with the spinal cord 14 than a non-deformable device.

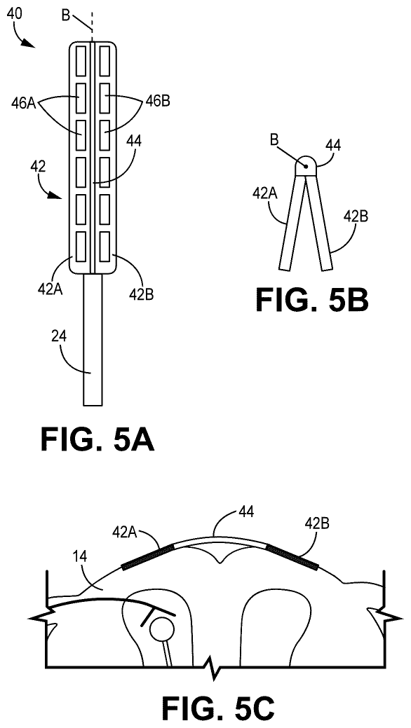

FIGS. 5A-5C depict an alternative spinal cord stimulation device 40 that is substantially similar to device 30 discussed above except as detailed herein. That is, the electrode body 42 has a deformation section 44 similar to deformation section 34 discussed above, along with two sides 42A, 42B of the body 42 on opposing sides of the deformation section 44 substantially similar to the two sides 32A, 32B discussed above. Except as detailed herein, the various components are substantially similar and have substantially similar functionality and features as the corresponding components in the embodiment depicted in FIGS. 4A-4C.

In this specific embodiment, the contacts 46A, 46B disposed along the length of the electrode body 42 are depicted in detail. More specifically, there are at least two contacts 46A disposed on the first side 42A and at least two contacts 46B disposed on the second side 42B. In the exemplary embodiment depicted in FIG. 5A, the first side 42A has six contacts 46A and the second side 42B has six contacts 46B.

In various implementations of this device, the electrode body 42 can have a width ranging from about 3 mm to about 5 mm, the thin section 44 can have a width ranging from about 1 mm to 3 mm, and the lead body 24 can have a width ranging from about 1 mm to about 3 mm. Further, the contacts 46A, 46B can be rectangular, oval, or any other known shape and can have a width ranging from about 1 mm to about 3 mm and a length ranging from about 3 mm to about 6 mm such that the contacts 46A, 46B can be disposed on each of the sides 42A, 42B. More specifically, the contacts 46A are disposed on the side 42A, while the contacts 46B are disposed on the side 42B. In addition, in certain implementations, two or more rows of contacts can be provided on both sides 42A, 42B of the electrode body 42.

FIG. 5C depicts the device 40 in use in a fashion similar to that described above with respect to the embodiment of FIG. 4C. More specifically, the electrode body 42 is positioned on the spinal cord 14 so that the body 42 is positioned over an area substantially similar to the area A depicted in FIGS. 2A and 2B as discussed above. Further, the body 42 is deformed at the deformation section 44 such that the two sides 42A, 42B conform to the shape of the spinal cord 14 such that the sides 42A, 42B are positioned in contact with the spinal cord 14 on both sides of the midline as shown, thereby ensuring that the body 42 is in better contact with the spinal cord 14 than a non-deformable device.

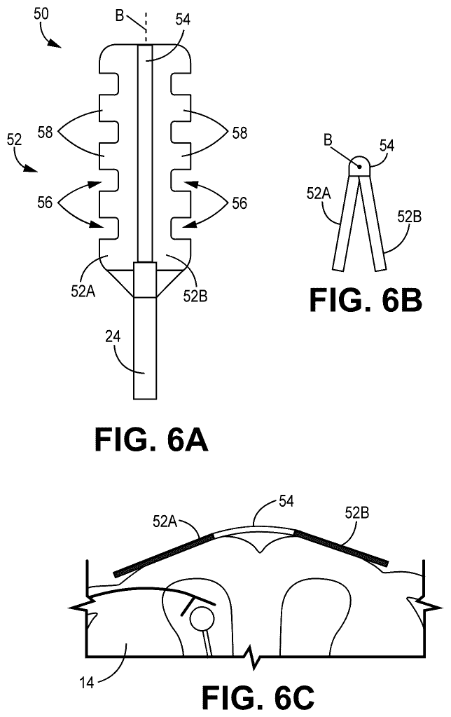

According to another embodiment, FIGS. 6A-6B depict a spinal cord stimulation device 50 having an electrode body 52 with a thin section 54 and two sides 52A, 52B, each containing at least one electrode (not shown) disposed thereon. Except as detailed herein, the various components are substantially similar and have substantially similar functionality and features as the corresponding components in the embodiments depicted in FIGS. 4A-4C and 5A-5C. In addition, the electrode body 52 also has a series of notches (also referred to as “slots”) 56 formed or otherwise defined along the outer edges of the sides 52A, 52B such that the notches 56 extend toward the thin section 54 and are transverse to the longitudinal axis B of the thin section 54. In one embodiment, the electrode body 52 can have at least two notches 56 defined in each side 52A, 52B of the body 52. Alternatively, the body 52 can have at least four notches 56 in each side 52A, 52B as shown. In a further embodiment, the body 52 can have any number of notches 56 in each side 52A, 52B. The notches 56 create a series of protrusions 58 that result in the sides 52A, 52B being more deformable than the sides 52A, 52B without such notches 56. Thus, the notches 56 further enhance the deformability and flexibility of the electrode body 52, thereby further enhancing the ability of the body 52 to conform to the shape of the target spinal cord such that the body 52 and contacts (not shown) can be in contact with the target spinal cord and remain in contact therewith or close proximity thereto even when the spinal cord moves.

In accordance with certain embodiments, the electrode body 52 can have a width ranging from about 7 mm to about 13 mm (not include the width of the notched portions), the thin section 54 can have a width ranging from about 1 mm to 3 mm, and the lead body 24 can have a width ranging from about 1 mm to about 3 mm. Further, each of the notches 56 can extend inward from the outer edge of the side 52A, 52B toward the thin section 54 an amount ranging from about 1 mm to about 4 mm (or, put another way, each of the protrusions 58 have a length ranging from about 1 mm to about 4 mm).

FIG. 6C depicts the device 50 in use in a fashion similar to that described above with respect to the embodiments of FIGS. 4C and 5C. More specifically, the electrode body 52 is positioned on the spinal cord 14 so that the body 52 is positioned over an area substantially similar to the area A depicted in FIGS. 2A and 2B as discussed above. Further, the body 52 is deformed at the deformation section 54 (and in part as a result of the notches 56) such that the two sides 52A, 52B conform to the shape of the spinal cord 14 such that the sides 52A, 52B are positioned in contact with the spinal cord 14 on both sides of the midline as shown, thereby ensuring that the body 52 is in better contact with the spinal cord 14 than a non-deformable device.

Certain implementations of the deformable electrode bodies 32, 42, 52 discussed above can also be deployed in a minimally invasive manner as a result of the deformable nature of the bodies 32, 42, 52. More specifically, any of the bodies 32, 42, 52 can be deformed into a collapsed or folded configuration similar to that shown in FIGS. 4B, 5B, and 6B. In such a configuration, the two sides (including any of 32A, 32B, 42A, 42B, or 52A, 52B) can be deformed such that the sides are folded toward each other a desired amount. For example, the two sides can be urged toward each other a slight amount, sufficiently so that the two sides are in contact with each other, or any amount in between. Thus, the two sides (including any of 32A, 32B, 42A, 42B, or 52A, 52B) can be folded or otherwise urged together such that they are positioned to form an acute angle with the sides disposed at any distance from each other, including the specific angle as shown. That is, the two sides can be disposed in relation to each other at any angle ranging from about 89° to about 0°. In this collapsed (or “folded”) configuration, the entire device having any type of electrode body 32, 42, 52 can be inserted through the lumen of a catheter or sheath to the target area adjacent to the patient's spinal cord.

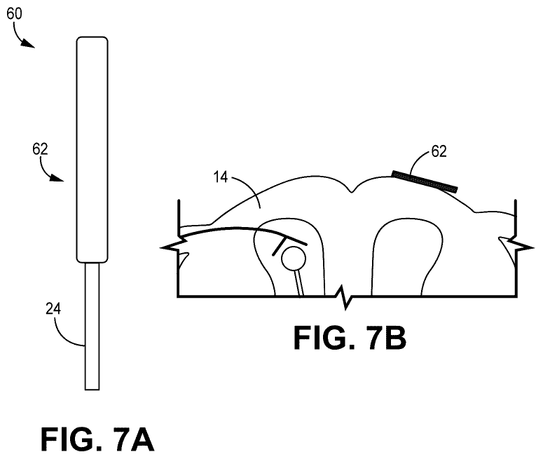

According to another embodiment as shown in FIGS. 7A and 7B, the spinal cord stimulation device 60 can have an electrode body 62 that is fairly narrow in comparison to the electrode bodies 32, 42, 52 discussed above such that the electrode body 62 is only as wide as or slightly wider than the lead body 24. That is, this body 62 can have a width ranging from about 1.5 mm to about 4 mm. (In contrast, the various lead bodies 32, 42, 52 can have different widths falling within the range of about 5 mm to about 13 mm as discussed in detail above.) Like the electrode bodies 32, 42, 52 discussed above, this electrode body 62 can also be positioned in contact with or adjacent to the target spinal cord and have sufficient flexibility along its length such that the body 62 can deform as a result of the deformation or movement of the spinal cord and remain in contact with the spinal cord while not causing any damage to the spinal cord or surrounding tissues (in comparison to a less flexible device which could cause such damage). In contrast to the previous bodies 32, 42, 52, the electrode body 62 does not need to have deformable sides that are deformable around the longitudinal axis A, because the body 62 is sufficiently narrow that no such deformability around that axis is needed.

In accordance with certain embodiments, the lead body 24 in FIG. 7A can have a width ranging from about 1 mm to about 3 mm.

FIG. 7B depicts the electrode body 62 in use. More specifically, the electrode body 62 is positioned on the spinal cord 14 so that the body 62 is positioned over a portion of the spinal cord 14 on one side or the other of the midline such that the body 62 positioned in contact with the spinal cord 14 on the desired side of the midline as shown. This positioning of the body 62 ensures that the body 62 is in better contact with the spinal cord 14 than a non-deformable device and is deformable such that the body 62 can conform along its length to the movement of the spinal cord 14.

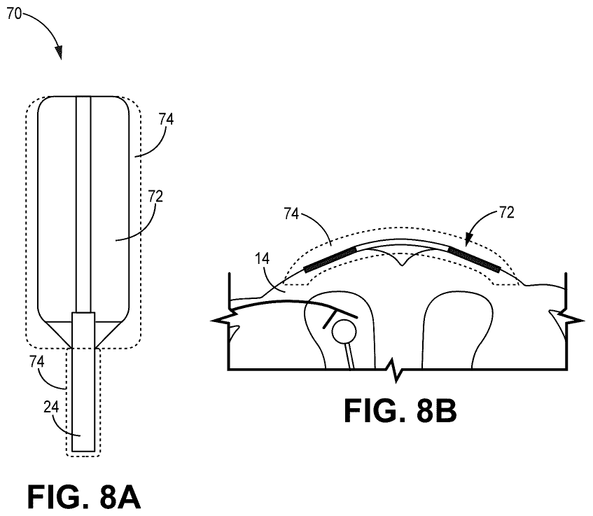

FIGS. 8A-8B depict an alternative spinal cord stimulation device 70 that is substantially similar to device embodiments discussed above except as detailed herein. That is, except as discussed herein, the various components are substantially similar and have substantially similar functionality and features as the corresponding components in the various device embodiments discussed above.

In this specific embodiment, the device 70 has a device body 72 and a lead body 24 that are covered or coated with a flexible coating or overmold 74. The coating 74 can be made of silicone or any other known biocompatible and/or bioresorbable flexible and/or rubber-like material that can be used in a coating for a neural or spinal electrode device. According to certain implementations, the device 70 with the coating 74 can have a maximum total thickness that does not exceed around 0.5 mm.

Any of the various device implementations disclosed or contemplated herein can have a coating or overmold 74 substantially similar to the coating 74 of device 70 discussed above.

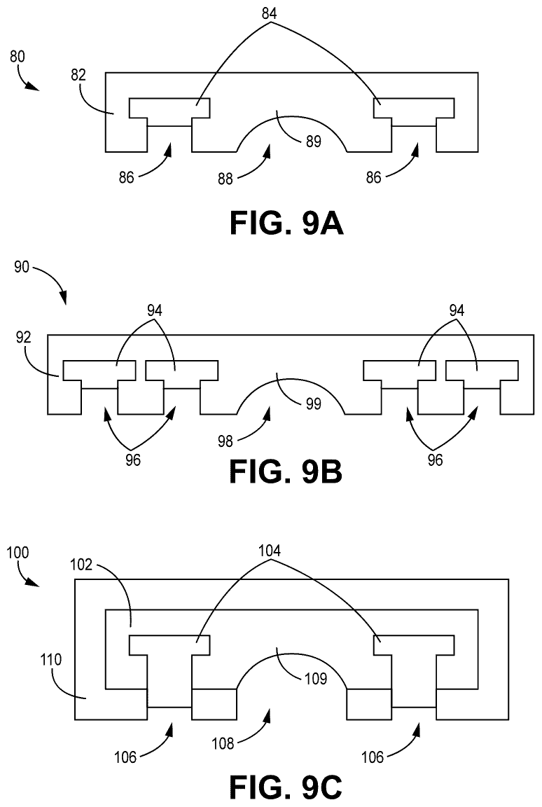

FIGS. 9A-9C depict various electrode body embodiments that can be incorporated into any of the device implementations disclosed or contemplated herein. More specifically, each of FIGS. 9A-9C depict a cross-section of an electrode body embodiment (wherein each body is viewed such that the longitudinal axis of the body extends out of the document). For example, FIG. 9A depicts an electrode body 80 having two rows of electrodes (with a single electrode 84 visible in each row) disposed within the insulation material 82, with one row disposed on each side of the deformation section 89. Further, access openings 86 are provided that provide access to the contacts 84 as shown. In certain embodiments, the openings 86 are formed via etching or any other method. In addition, the body 80 has a channel 88 formed in the body 80 that results in the deformation section 89 in the body 80. In certain embodiments, the channel 88 can be formed via etching or any other method.

Further, FIG. 9B depicts an electrode body 90 having four rows of electrodes (with a single electrode 94 visible in each row) disposed within the insulation material 92, with two rows disposed on each side of the deformation section 99. Further, access openings 96 are provided that provide access to the contacts 94 as shown. In certain embodiments, the openings 96 are formed via etching or any other method. In addition, the body 90 has a channel 98 formed in the body 90 that results in the deformation section 99 in the body 90. In certain embodiments, the channel 98 can be formed via etching or any other method.

Further, FIG. 9C depicts an electrode body 100 having two rows of electrodes (with a single electrode 104 visible in each row) disposed within the insulation material 102, with one row disposed on each side of the deformation section 109. Further, access openings 106 are provided that provide access to the contacts 104 as shown. In certain embodiments, the openings 106 are formed via etching or any other method. In addition, the body 100 has a channel 108 formed in the body 100 that results in the deformation section 109 in the body 100. In certain embodiments, the channel 108 can be formed via etching or any other method. Further, an overmold or coating 110 is disposed over the body 100 as shown.

The various deformation sections described herein with respect to any of the various embodiments disclosed or contemplated herein can be formed via a similar channel structure as depicted in FIGS. 9A-9C. Alternatively, any of the deformation sections can be any known structure that increases the deformability of the body at the deformation section, including a trough, furrow, recess, or the like.

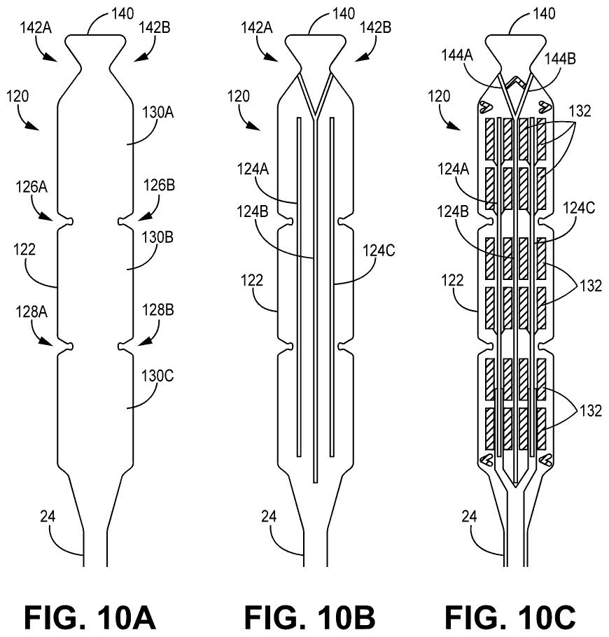

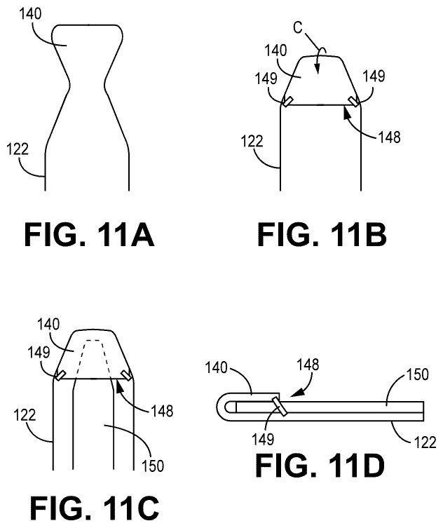

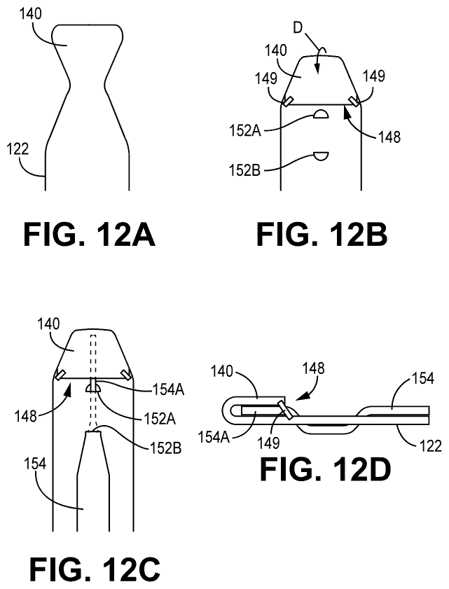

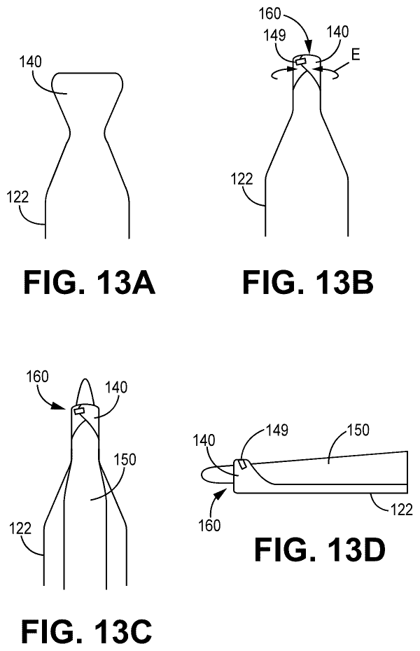

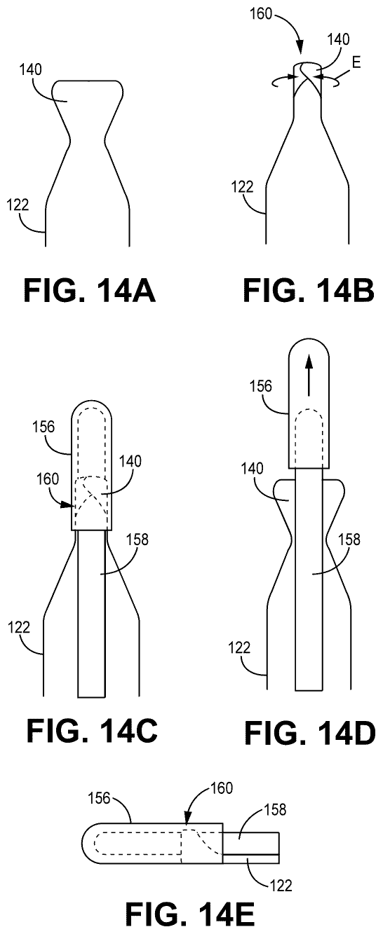

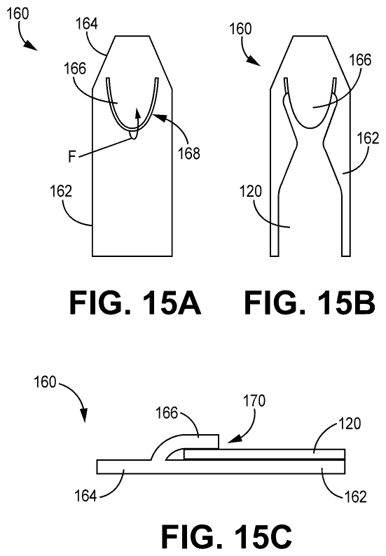

FIGS. 10A-10C depict a further implementation of a deformable thin film spinal cord stimulation device 120 having an electrode body 122. Except as detailed herein, the various components are substantially similar and have substantially similar functionality and features as the corresponding components in the embodiments depicted in FIGS. 4A-6C and discussed above. In this specific embodiment, the electrode body 122 has three deformation sections 124A, 124B, 124C that extend longitudinally along the length of the body 122 as shown that are thinner than the rest of the body 122. According to certain implementations, the three deformation sections 124A, 124B, 124C can increase the deformability of the body 122 (such that opposing sides of the body 122 can be more easily urged toward each other as discussed above) in comparison to one or two deformation sections. More specifically, the body 122 is deformable around the axis of each of the three deformation sections 124A, 124B, 124C in fashion similar to that described above with respect to the embodiments with a single deformation section, thereby resulting in increased deformability by comparison. Alternatively, the body 122 can have at least two deformation sections, four deformation sections, five deformation sections, six deformation sections, seven deformation sections, eight deformation sections, nine deformation sections, ten deformation sections, or any number of deformation sections disposed in the body 122.

In addition, as best shown in FIG. 10A, in some implementations, the electrode body 122 also has two pairs of notches (also referred to as “slots”) 126A-B, 128A-B formed or otherwise defined along the elongate outer edges of the body 122. In this embodiment, each pair of notches 126A-B, 128A-B is made up of two notches 126A-B, 128A-B disposed across from each other on opposite sides of the body 122. The opposing notches of each pair 126A-B, 128A-B extend inward toward the center of the body 122 and are transverse to the longitudinal axis of the body 122. In the exemplary embodiment as shown, the electrode body 122 has two notch pairs 126A-B, 128A-B. Alternatively, the body 122 can have at least one pair, three pairs, four pairs, five pairs, six pairs, seven pairs, eight pairs, nine pairs, ten pairs, or any number of pairs of notches defined on opposite sides of the body 122. In this implementation, each notch 126A-B, 128A-B has curved sides such that the width of the notch decreases as the depth of the notch increases. In other words, each notch is narrower at its deepest point than it is at or near the edge of the body 122.

The notch pairs 126A-B, 128A-B, according to one embodiment, are strain-relief features that enhance the deformability of the body 122 in comparison to a body 122 without the notch pairs. As best shown in FIG. 10A, the two notch pairs 126A-B, 128A-B form three body sections 130A, 130B, 130C along the length of the body 120. That is, the notches 126A-B, 128A-B create structural separation between different portions of the body 120, thereby resulting in three different body sections 130A-C as shown with each body section 130A-C having some structural independence in comparison to the other body sections 130A-C. As a result, the notch pairs 126A-B, 128A-B allow for the body 122 to flex in either direction parallel with the plane of the document on which the body 122 is depicted while also allowing the body 122 to flex in either direction transverse to the plane of the document. Thus, the notches 126A-B, 128A-B further enhance the deformability and flexibility of the electrode body 122, thereby further enhancing the ability of the body 122 to conform to the shape of the target spinal cord such that the body 122 and contacts 132 (as discussed in detail below) can be in contact with the target spinal cord and remain in contact therewith or close proximity thereto even when the spinal cord moves.

Further, in accordance with certain implementations as best shown in FIG. 100, the electrode body 122 can have 24 contacts 132 disposed along the length of the body 122. In this exemplary embodiment, the contacts 132 are disposed in rows: six rows extending “horizontally” across the width of the body 122 and four rows extending “vertically” along the length of the body 122. Thus, each horizontal row has a contact 132 disposed between the left edge and the first deformation section 124A, a contact 132 disposed between the first 124A and second 124B deformation sections, a contact 132 disposed between the second 124B and third 124C deformation sections, and a contact disposed between the third deformation section 124C and the right edge of the body 122. Alternatively, the body 122 can have any number of contacts 132 in any known configuration. That is, the body 122 can have one, two, three, four, five, six, seven, eight, nine, 10, 11, 12, 13, 14, 15, 16, 17, 18, 19, 20, 21, 22, 23, 25, 26, 27, 28, 29, 30, 31, 32, or any number of contacts 132 in any configuration.

As such, as best shown in FIGS. 4C, 5C, 6C, 7B, and 8B, the deformability of the bodies 32, 42, 52, 62, 72 (along with bodies 80, 90, 100, 120) can allow for those bodies 32, 42, 52, 62, 72 (along with bodies 80, 90, 100, 120) to bend around the outer curvature of the spinal cord 14, bend laterally with the spinal cord, and/or flex longitudinally along with the spinal cord 14 such that the bodies 32, 42, 52, 62, 72, 90, 100, 120 maintain better contact with the spinal cord 14 than non-deformable devices.

According to certain alternative embodiments, the electrode body 122—or any of the electrode bodies according to any of the other embodiments herein—can also have a distal flap 140 disposed at a distal end of the body 122. In certain implementations, the flap 140 is defined by two V-shaped notches 142A, 142B that are defined in a distal portion of the body 122 such that the notches 142A, 142B define the outer edges of the flap 140 as shown. Further, in certain optional embodiments as best shown in FIG. the body 122 also has two distal deformation sections 144A, 144B, each of which extends from the distal end of the second deformation section 124B at an angle to one of the two V-shaped notches 142A, 142B as shown. Alternatively, the two distal deformation sections 144A, 144B can extend from the distal end of the second deformation section 124B to any point along the outer edge of the body 122 at some point between the flap 140 and the rest of the body 122. In a further alternative, the distal deformation section can be a single deformation section extending from one side of the body 122 to the other at some point between the flap 140 and the rest of the body 122.

In accordance with certain implementations, the V-shaped notches 142A, 142B and the distal deformation sections 144A, 144B create structural separation between the body 122 and the flap 140, thereby resulting in the flap 140 being able to more easily flex or fold in relation to the body 122 such that the flap 140 can be used to create three-dimensional structures at the distal end of the body 122. In some embodiments, the three-dimensional structures can be used to assist with the use of a stylet, pusher, or other insertion device to implant any deformable spinal cord stimulation device embodiment herein in a minimally-invasive fashion.

For example, FIGS. 11A-14E depict the different three-dimensional structures that can be formed via the flap 140, according to various embodiments. More specifically, FIGS. 11A-11D depict the flap 140 being foldable to form a pocket 148 into which a known pusher 150 can be inserted to assist with inserting the device into an area adjacent to a target spinal cord. More specifically, as best shown in FIGS. 11B and 11D, the flap 140 is folded back onto the body 122 as shown via arrow C such that a pocket 148 is formed between the flap 140 and the body 122. Once the pocket 148 is formed as desired, the flap 140 can be attached to the body 122 via any known attachment mechanisms 149. In use, the known pusher 150 can be urged into the pocket 148 as best shown in FIGS. 11C and 11D such that the pusher 150 and electrode body 122 (and entire device) can be urged into the patient in a minimally-invasive procedure to position the device adjacent to the patient's spinal cord as discussed elsewhere herein. The known pusher 150—including any known pusher discussed herein with respect to any other embodiments—can be any known stylet or other pushing device for implanting a spinal cord stimulation device.

FIGS. 12A-12D depict an alternative embodiment similar to the embodiment of FIGS. 11A-11D except that the body 122 includes two openings 152A-B to further assist with the implantation method. More specifically, in addition to the flap 140 being folded back onto the body 122 as shown via arrow D such that a pocket 148 is formed to receive the pusher 154, the body 122 also has two openings 152A-B configured to receive the stylet-style distal tip 154A of the pusher 154. In use, the known pusher 154 can be urged through the openings 152A-B as best shown in FIGS. 12C and 12D and into the pocket 148 such that the pusher 154 and electrode body 122 (and entire device) can be urged into the patient in a minimally-invasive procedure to position the device adjacent to the patient's spinal cord as discussed elsewhere herein.

In another alternative implementation, FIGS. 13A-13D depict the flap 140 being foldable to form a collar 160 into which the known pusher 150 can be inserted to assist with inserting the device into an area adjacent to a target spinal cord. More specifically, as shown in FIG. 13B, the opposing corners of the flap 140 are folded onto each other as shown via arrows E such that a collar 160 is formed between the flap 140 and the body 122. Once the collar 160 is formed as desired, the flap 140 can be attached to itself via any known attachment mechanism 149. In use as best shown in FIGS. 13C and 13D, the known pusher 150 can be urged into the collar 160 such that the pusher 150 and electrode body 122 (and entire device) can be urged into the patient in a minimally-invasive procedure to position the device adjacent to the patient's spinal cord as discussed elsewhere herein.

FIGS. 14A-14E depict an alternative embodiment similar to the embodiment of FIGS. 13A-13D except that the flap 140 is retained in the shape of the collar 160 by a cap 156 positioned thereover (rather than an attachment mechanism). More specifically, in addition to the flap 140 being folded to form a collar 160 into which the known pusher 150 can be inserted as described in detail above, a cap 156 or similar structure is provided that can be placed over the distal tip of the pusher 158 and the flap 140 to help retain the flap 140 in the collar 160 structure (instead of any attachment mechanism(s)). In use as best shown in FIGS. 14C-14E, the known pusher 150 can be urged into the collar 160, the cap 156 can be positioned over the collar 160, and the pusher 158 can then be positioned through collar 160 within the cap 156 such that the pusher 150, electrode body 122 (and entire device), and cap 160 can be urged into the patient in a minimally-invasive procedure to position the device adjacent to the patient's spinal cord as discussed elsewhere herein. Once the body 122 (and entire device) is positioned as desired, the pusher 158 can be advanced further distally such that the cap 156 is urged distally from the flap 140, thereby allowing the flap 140 to its original flat configuration as shown in FIG. 14D.

In accordance with other implementations, a pusher device 160 is provided that can be used to deliver any of the various deformable spinal cord stimulation device embodiments herein to the target area of a patient's spinal cord. The device 160 has an elongate pusher body 162, a distal tip 164, and a deployable pusher flap 166 formed into the body 162 as shown. In one implementation, the flap 166 is created via a slit or other type of gap/opening 168 defined in the body 162 such that the flap 166 can be urged upward away from the body 168 as shown with arrow F in FIG. 15A. In accordance with certain implementations, the flap 166 is made of a shape-memory material (such as nitinol or the like) such that force must be applied to urge the flap 166 away from the body 162 and further such that when the force is removed, the flap 166 returns to its natural state (flush with the body 162). In certain implementations, the body 162 is made of the same material as the flap 166. Alternatively, the body 162 can be made of any known material typically used in pusher devices typically used for implantation of spinal cord stimulation devices.

In use, any of the deformable spinal cord stimulation device embodiments disclosed or contemplated herein can be inserted using the pusher device 160. More specifically, as best shown in FIGS. 15B and 15C, the flap 166 is raised upward from the body 162 and the distal end of a stimulation device (such as the electrode body 120 of the device depicted in FIGS. 10A-10C) is inserted underneath the flap 166 such that the flap 166 creates a slot 170 in which the body 120 is positioned as shown. Once the stimulation device 120 is positioned as desired, the combination of the pusher device 160 and the stimulation device 120 can be urged distally into the target area of the patient's spinal cord. Once the stimulation device 120 is positioned as desired, the pusher device 160 can be urged distally in relation to the stimulation device 120 (while the stimulation device 120 is maintained in position) until the distal end of the stimulation device 120 is no longer positioned under the flap 166. At this point, the flap 166 returns to its natural position and the pusher device 160 is retracted proximally from the target area and out of the patient.

While the various systems described above are separate implementations, any of the individual components, mechanisms, or devices, and related features functionality, and/or dimensions within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A spinal cord stimulation device comprising:
(a) an elongate thin film lead body; and
(b) a thin film electrode body disposed at one end of the elongate thin film lead body, the thin film electrode body comprising:
(i) at least one deformation section disposed longitudinally through the electrode body such that the at least one deformation section is parallel with a longitudinal axis of the lead body; and
(ii) at least two contacts disposed on the electrode body.

2. The device of claim 1, wherein the at least two contacts comprises at least twelve contacts.

3. The device of claim 1, wherein the at least one deformation section comprises at least two deformation sections.

4. The device of claim 1, wherein the at least one deformation section comprises three deformation sections.

5. The device of claim 1, wherein the thin film electrode body comprises a distal flap disposed at a distal end of the thin film electrode body.

6. The device of claim 5, further comprising at least one distal deformation section comprising a first end disposed at a distal end of the at least one deformation section and a second end disposed at a side of the electrode body.

7. The device of claim 6, wherein the second end of the at least one distal deformation section is disposed between the distal flap and the distal end of the thin film electrode body.

8. The device of claim 5, wherein the flap is moveable between a flat configuration and a pocket configuration.

9. The device of claim 5, wherein the flap is moveable between a flat configuration and a collar configuration.

10. A spinal cord stimulation device comprising:
(a) an elongate thin film lead body; and
(b) a thin film electrode body disposed at one end of the elongate thin film lead body, the thin film electrode body comprising:
(i) at least two deformation sections disposed longitudinally through the electrode body;
(ii) at least one first contact disposed between a first outer edge of the electrode body and a first of the at least two deformation sections;
(iii) at least one second contact disposed between the first and a second of the at least two deformation sections; and
(iv) at least one third contact disposed between the second of the at least two deformation sections and a second outer edge of the electrode body,
wherein the first and second outer edges are moveable in relation to each other via the at least two deformation sections such that the electrode body is laterally conformable to a shape of a target spinal cord.

11. The device of claim 10, further comprising at least one pair of notches defined in the first and second sides of the electrode body, whereby the electrode body has increased lateral and longitudinal flexiblity.

12. The device of claim 10, wherein the first and second outer edges are rotatable around a longitudinal axis of the electrode body.

13. The device of claim 10, wherein the thin film electrode body comprises a distal flap disposed at a distal end of the thin film electrode body.

14. The device of claim 13, wherein the flap is moveable between a flat configuration and a pocket configuration or a collar configuration.

15. A spinal cord stimulation device comprising:
(a) an elongate thin film lead body;
(b) a thin film electrode body disposed at one end of the elongate thin film lead body, the thin film electrode body comprising:
(i) at least three deformation sections disposed longitudinally through the electrode body such that each of the at least three deformation sections is parallel with a longitudinal axis of the lead body;
(ii) at least two contacts disposed on the electrode body; and
(iii) first and second outer edges moveable in relation to each other via the at least three deformation sections such that the electrode body is laterally conformable to a shape of a target spinal cord; and
(c) a distal flap disposed at a distal end of the thin film electrode body, wherein the flap is movable between a flat configuration and a pocket configuration or a collar configuration.

16. The device of claim 15, further comprising at least one distal deformation section comprising a first end disposed at a distal end of one of the at least three deformation sections and a second end disposed at one of the first and second outer edges.

17. The device of claim 16, wherein the second end of the at least one distal deformation section is disposed between the distal flap and the distal end of the thin film electrode body.

18. A method of implanting a deformable spinal cord stimulation device, the method comprising:
preparing the deformable spinal cord stimulation device for implantation, wherein the deformable spinal cord stimulation device comprises:
(a) an elongate thin film lead body;
(b) a thin film electrode body disposed at one end of the elongate thin film lead body, the thin film electrode body comprising:
(i) at least one deformation section disposed longitudinally through the electrode body such that the at least one deformation section is parallel with a longitudinal axis of the lead body; and
(ii) at least two contacts disposed on the electrode body; and
(c) a distal flap disposed at a distal end of the thin film electrode body, wherein the flap is movable between a flat configuration and a pusher receiving configuration;
urging the distal flap into the pusher receiving configuration;
inserting a distal end of a pusher device into the pusher receiving configuration; and
urging the deformable spinal cord stimulation device into a target area of a patient's spinal cord with the pusher device.

19. The method of claim 18, wherein the at least one deformation section comprises at least two deformation sections.

20. The method of claim 18, wherein the pusher receiving configuration comprises a pocket configuration or a collar configuration.
